(19) United States
(12) Patent Application Publication
(10) Pub. No.: US 2008/0108614 A1
(43) Pub. Date: May 8, 2008

(54) Substituted 2-Amino-Quinazolin-4-Cn Compounds for Use in the Treatment of Cns Disorders, Pain, Stroke, Addiction and Epilepsy, their Preparation and Use as Intermediates

(76) Inventors: Pedro Noheda-Marin, Madrid (ES); Nuria Tabares-Cantero, Madrid (ES); Raul Benito-Arela, Madrid (ES); Sergio Maroto Quintana, Madrid (ES); Luis Miguel Lozano Gordillo, Madrid (ES)

Correspondence Address: COOPER & DUNHAM, LLP, 1185 AVENUE OF THE AMERICAS, NEW YORK, NY, 10036, US

(21) Appl. No.: 11/794,882

(22) PCT Filed: Jan. 9, 2006
(86) PCT No.: PCT/EP06/00096

§ 371 Date: Dec. 17, 2007

(30) Foreign Application Priority Data
Jan. 7, 2005 (EP) 05000179.1

(51) Int. Cl. A61K 31/517; A61P 25/00; C07D 239/95; C07D 403/04; C07D 413/04; A61K 31/5377

(52) U.S. Cl. 514/234.5; 544/287; 544/116; 514/266.3; 514/252.17

(57) ABSTRACT

The present invention relates to substituted quinazoline compounds, methods for their preparation, as well as their use as intermediates for the preparation of active biomolecules.

The present invention relates to substituted quinazoline compounds, methods for their preparation, as well as their use as intermediates for the preparation of active biomolecules.

Quinazolins are compounds of high interest due to the activity of the compounds which could be prepared starting from them including e.g. saxitoxin.

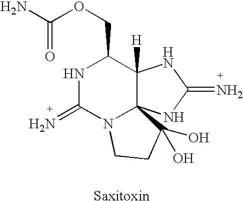

Saxitoxin is—according to the Merck Index on CD Version 12:1—a mussel poison; clam poison; paralytic shellfish poison; gonyaulax toxin. This powerful neurotoxin is produced by the dinoflagellates Gonyaulax catenella, or G. tamarensis, the consumption of which causes the California sea mussel Mytilus californianus, the Alaskan butterclam Saxidomus giganteus and the scallop to become poisonous: Sommer et al., Arch. Pathol. 24, 537, 560 (1937); Schantz et al., Can. J. Chem. 39, 2117 (1961); Ghazarossian et al., Biochem. Biophys. Res. Commun. 59, 1219 (1974). These poisonous shellfish have been connected to instances of toxic “red-tides” where the high concentrations of algae discoloring the water were of the Gonyaulax genus. Isoln and partial characterization: Schantz et al., J. Am. Chem. Soc. 79, 5230 (1957); Mold et al., ibid. 5235. Saxitoxin is a very popular tool in neurochemical research and as a sodium channel blocker is recently being described in a number of therapeutic uses.

Thus, in one of its aspects the present invention relates to substituted quinazoline compound of general formula I,

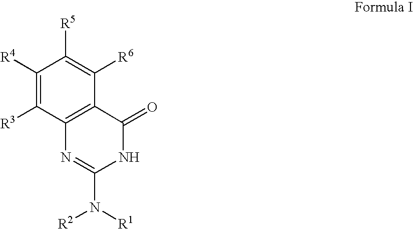

wherein
R¹ and R² independently of one another represent hydrogen; C₁₋₄-alkyl, with alkyl being linear or branched, saturated or unsaturated, substituted or unsubstituted; or an appropriate protective group;
or R¹ and R² together with the Nitrogen they both bind to form a heterocyclic ring or an appropriate protective group;
R³ represents halogen, OH or O—C₁₋₄-alkyl, with alkyl being linear or branched, saturated or unsaturated, substituted or unsubstituted; or O—P, with P being an appropriate protective group;
at least one of R⁴ and R⁵ represents halogen; OH; O—C₁₋₄-alkyl, with alkyl being linear or branched, saturated or unsaturated, substituted or unsubstituted; or O—P, with P being an appropriate protective group; while the other represents hydrogen; OH; halogen; O—C₁₋₄-alkyl, with alkyl being linear or branched, saturated or unsaturated, substituted or unsubstituted; or O—P, with P being an appropriate protective group;
R⁶ represents hydrogen or C(O)—NR⁷R⁸;
R⁷ and R⁸ independently of one another represent hydrogen; C₁₋₄-alkyl, with alkyl being linear or branched, saturated or unsaturated, substituted or unsubstituted; or an appropriate protective group;
or R⁷ and R⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

These compounds are very useful as intermediates for the synthesis of biomolecules and in addition seem to have a quite surprising effect as sodiumchannel blockers themselves.

As a general remark the claim to the compounds will cover as well any prodrug of the claimed and invented compounds as well as any use thereof especially including their esters and ethers. Examples of well known methods of producing a prodrug of a given acting compound are known to those skilled in the art and can be found e.g. in Krogsgaard-Larsen et al., Textbook of Drugdesign and Discovery, Taylor & Francis (April 2002).

The expression “an appropriate protective group” is defined as a chemical group blocking a reactive site, e.g. hydroxyl groups or amino groups, from taking part in a chemical reaction. The appropriate protective groups are known to the skilled chemist and can be found in literature. Especially, in this application this relates to the protective groups described in Greene and Wuts “Protective Groups in Organic Synthesis”, Third Edition, 1999, John Wiley & Sons Inc. included hereby in its entirety by reference. Preferred protective groups include tert-butoxycarbonyl (Boc), benzyloxycarbonyl (Cbz), trifluoroacetyl (TFA) or 9-fluorenylmethoxycarbonyl (Fmoc), phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP), Dithiasuccinimide (Dts), tert-butyl, acetyl or benzoyl including in all cases their structurally related analogs.

In the context of this invention, alkyl and cycloalkyl radicals are understood as meaning saturated and unsaturated (but not aromatic), branched, unbranched and cyclic hydrocarbons, which can be unsubstituted or mono- or polysubstituted. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C₁₋₁₀-alkyl and C₁₋₁₈-alkyl represents C1-, C2-, C3-, C4-, C5-, C6-, C7-, C8-, C9-, C10-, C10-, C12-, C13-, C14-, C15-, C16-, C17- or C18-alkyl. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. The alkyl and cycloalkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl, (if substituted also CHF₂, CF₃ or CH₂OH) as well as pyrazolinone, oxopyrazolinone, [1,4]-dioxane or dioxolane. Preferred linear or branched, saturated or unsaturated aliphatic groups/alkyl radicals, which may be substituted by one or more substituents, may preferably be selected from the group consisting of methyl, ethyl, n-propyl, isopropyl, n-butyl, iso-butyl, sec-butyl, tert-butyl, n-pentyl, n-hexyl, n-heptyl, n-octyl, n-nonyl, n-decyl, vinyl, ethinyl, propenyl, propinyl, butenyl and butinyl.

Here, in connection with alkyl and cycloalkyl—unless expressly defined otherwise—the term substituted in the context of this invention is understood as meaning replacement of at least one hydrogen radical by F, Cl, Br, I, NH₂, SH or OH, “polysubstituted” radicals being understood as meaning that the replacement takes effect both on different and on the same atoms several times with the same or different substituents, for example three times on the same C atom, as in the case of CF₃, or at different places, as in the case of —CH(OH)—CH═CH—CHCl₂. Particularly preferred substituents here are F, Cl and OH. In respect of cycloalkyl, the hydrogen radical can also be replaced by OC₁₋₃-alkyl or C₁₋₃-alkyl (in each case mono- or polysubstituted or unsubstituted), in particular methyl, ethyl, n-propyl, i-propyl, CF₃, methoxy or ethoxy.

The term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂—, etc.

An aryl radical is understood as meaning ring systems with at least one aromatic ring but without heteroatoms even in only one of the rings. Examples are phenyl, naphthyl, fluoranthenyl, fluorenyl, tetralinyl or indanyl, in particular 9H-fluorenyl or anthracenyl radicals, which can be unsubstituted or monosubstituted or polysubstituted.

A heterocyclyl radical is understood as meaning heterocyclic ring systems which contain one or more heteroatoms from the group consisting of nitrogen, oxygen and/or sulfur and can also be mono- or polysubstituted. The heterocyclic ring systems may consist of condensed rings and may be fully or just in a part of the condensed rings saturated or unsaturated or even aromatic. A subgroup of the heterocyclylc radicals/heterocyclyls are the heteroaryls/heteroaromatic radicals which contain at least one aromatic ringsystem. Included examples from the group of heterocyclyl radicals are pyrrolidine, pyrazolidine, triazolidine, piperidine, dithiolane, tetrahydrothiophene, tetrahydrofuran, dioxolane, dioxane, tetrahydropyran. Examples from the group of heteroaryl radicals/heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline.

A radical defined as a C(O)—NR⁷R⁸ group means

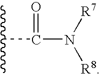

Here, in connection with aryl and heterocyclyl, substituted is understood as meaning substitution of the aryl or heteroaryl by R, OR, a halogen, preferably F and/or Cl, a CF₃, a CN, an NO₂, an NRR, a C₁₋₆-alkyl (saturated), a C₁₋₆-alkoxy, a C₃₋₈-cycloalkoxy, a C₃₋₈-cycloalkyl or a C₂₋₆-alkylene.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

These physiologically acceptable salts can also be formed with anions or acids in the context of this invention is understood as meaning salts of at least one of the compounds used according to the invention—usually protonated, for example on the nitrogen—as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The term “solvate” according to this invention is to be understood as meaning any form of the active compound according to the invention in which this compound has attached to it via non-covalent binding another molecule (most likely a polar solvent) especially including hydrates and alcoholates, e.g. methanolate. Solvates, preferably hydrates, of the compounds according to the invention may also be obtained by standard procedures known to those skilled in the art.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon or ¹⁵N-enriched nitrogen are within the scope of this invention.

N-oxides of the compounds according to the invention may also be obtained by standard procedures known to those skilled in the art.

The purification and isolation of the compounds according to the invention, of a corresponding stereoisomer, or salt, or solvate or any intermediate thereof may, if required, be carried out by conventional methods known to those skilled in the art, e.g. chromatographic methods or recrystallization.
In a preferred embodiment of the invention for the compound according to the invention according to formula I
R¹ and R² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R¹ and R² together with the Nitrogen they both bind to form a heterocyclic ring of the following formula:

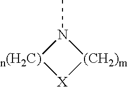

with n being 1, 2, 3 or 4, m being 1, 2, 3 or 4 and (n+m) being ≦6 and X being selected from S, O, NR⁹ or CHR⁹ with R⁹ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R³ represents halogen, OH or O—C₁₋₄-alkyl, with alkyl being linear or branched, saturated or unsaturated, unsubstituted or substituted by F, Cl, Br, I, NH₂, SH or OH; or O—P, with P being an appropriate protective group selected from tert-butyl, acetyl or benzoyl;
at least one of R⁴ and R⁵ represents halogen; OH; O—C₁₋₄-alkyl, with alkyl being linear or branched, saturated or unsaturated, unsubstituted or substituted by F, Cl, Br, I, NH₂, SH or OH; or O—P, with P being an appropriate protective group, selected from tert-butyl, acetyl or benzoyl; while the other represents hydrogen; OH; halogen; O—C₁₋₄-alkyl, with alkyl being linear or branched, saturated or unsaturated, unsubstituted or substituted by F, Cl, Br, I, NH₂, SH or OH; or O—P, with P being an appropriate protective group, selected from tert-butyl, acetyl or benzoyl.
R⁶ represents hydrogen or C(O)—NR⁷R⁸;
R⁷ and R⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R⁷ and R⁸ together with the Nitrogen they both bind to form a heterocyclic ring of the following formula:

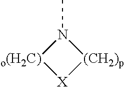

with o being 1, 2, 3 or 4, p being 1, 2, 3 or 4 and (o+p) being ≦6 and Y being selected from S, O, NR¹⁰ or CHR¹⁰ with R¹⁰ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

In another preferred embodiment of the invention for the compound according to the invention according to formula I
R¹ and R² independently of oneanother represent hydrogen; C₁₋₄-alkyl, with alkyl being linear or branched, saturated or unsaturated, substituted or unsubstituted;
R³ represents halogen, OH or O—C₁₋₄-alkyl, with alkyl being linear or branched, saturated or unsaturated, substituted or unsubstituted;
at least one of R⁴ and R⁵ represents halogen; OH; O—C₁₋₄-alkyl, with alkyl being linear or branched, saturated or unsaturated, substituted or unsubstituted;
   while the other represents hydrogen; OH; halogen; O—C₁₋₄-alkyl, with alkyl being linear or branched, saturated or unsaturated, substituted or unsubstituted;
R⁶ represents hydrogen or C(O)—NR⁷R⁸;
R⁷ and R⁸ independently of oneanother represent hydrogen; C₁₋₄-alkyl, with alkyl being linear or branched, saturated or unsaturated, substituted or unsubstituted.

In another preferred embodiment of the invention for the compound according to the invention according to formula I
R¹ and R² independently of oneanother represent hydrogen; C₁₋₄-alkyl, with alkyl being linear or branched, saturated and unsubstituted; and/or
R³ represents halogen, OH or O—C₁₋₄-alkyl, with alkyl being linear or branched, saturated and unsubstituted; and/or
at least one of R⁴ and R⁵ represents halogen; OH; O—C₁₋₄-alkyl, with alkyl being linear or branched, saturated and unsubstituted; while the other represents hydrogen; halogen; O—C₁₋₄-alkyl, with alkyl being linear or branched, saturated and unsubstituted; and/or
R⁶ represents hydrogen or C(O)—NR⁷R⁸;
R⁷ and R⁸ independently of oneanother represent hydrogen; C₁₋₄-alkyl, with alkyl being linear or branched, saturated and unsubstituted.

In another preferred embodiment of the invention for the compound according to the invention according to formula I halogen means Cl or F.

In another preferred embodiment of the invention for the compound according to the invention according to formula I neither of R³, R⁴ or R⁵ represent OH.

In a very preferred embodiment of the invention the compound according to the invention is a compound according to formula II

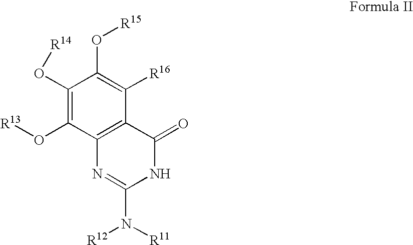

wherein
R¹¹ and R¹² independently of oneanother represent hydrogen; C₁₋₄-alkyl, with alkyl being linear or branched, saturated or unsaturated, substituted or unsubstituted; or an appropriate protective group;
or R¹¹ and R¹² together with the Nitrogen they both bind to form a heterocyclic ring or an appropriate protective group;
R¹³, R¹⁴ and R¹⁵ independently of oneantother represent hydrogen; C₁₋₄-alkyl, with alkyl being linear or branched, saturated or unsaturated, substituted or unsubstituted; or an appropriate protective group;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, substituted or unsubstituted; or an appropriate protective group;
or R¹⁷ and R¹⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

In a preferred embodiment of the invention for the compound according to the invention according to formula II
R¹¹ and R¹² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R¹¹ and R¹² together with the Nitrogen they both bind to form a heterocyclic ring of the following formula:

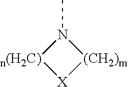

with n being 1, 2, 3 or 4, m being 1, 2, 3 or 4 and (n+m) being ≦6 and X being selected from S, O, NR¹⁹ or CHR¹⁹ with R¹⁹ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R¹³, R¹⁴ and R¹⁵ independently of oneanother represent hydrogen or C₁₋₄-alkyl, with alkyl being linear or branched, saturated or unsaturated, unsubstituted or substituted by F, Cl, Br, I, NH₂, SH or OH; or an appropriate protective group selected from tert-butyl, acetyl or benzoyl;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R¹⁷ and R¹⁸ together with the Nitrogen they both bind to form a heterocyclic ring of the following formula:

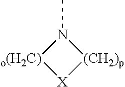

with o being 1, 2, 3 or 4, p being 1, 2, 3 or 4 and (o+p) being ≦6 and Y being selected from S, O, NR²⁰ or CHR²⁰ with R²⁰ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

In another preferred embodiment of the invention for the compound according to the invention according to formula II
R¹¹ and R¹² independently of oneanother represent hydrogen; C₁₋₄-alkyl, with alkyl being linear or branched, saturated or unsaturated, substituted or unsubstituted;
R¹³, R¹⁴ and R¹⁵ independently of oneantother represent hydrogen; C₁₋₄-alkyl, with alkyl being linear or branched, saturated or unsaturated, substituted or unsubstituted;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, substituted or unsubstituted.
In another preferred embodiment of the invention for the compound according to the invention according to formula II
R¹¹ and R¹² independently of oneanother represent hydrogen; C₁₋₄-alkyl, with alkyl being linear or branched, saturated and unsubstituted; and/or
R¹³, R¹⁴ and R¹⁵ independently of oneantother represent hydrogen; C₁₋₄-alkyl, with alkyl being linear or branched, saturated and unsubstituted; and/or
R¹⁶ represents hydrogen or C(O)—NR¹⁷R¹⁸;
with R¹⁷ and R¹⁸ independently of oneanother represent hydrogen; C₁₋₄-alkyl, with alkyl being linear or branched, saturated and unsubstituted.
In another preferred embodiment of the invention for the compound according to the invention according to formula II
R¹³, R¹⁴ and R¹⁵ independently of oneanother represent hydrogen or methyl, preferably
R¹³, R¹⁴ and R¹⁵ all represent hydrogen, or
R¹³, R¹⁴ and R¹⁵ all represent methyl.

In another preferred embodiment of the invention the compound according to the invention according to formula II is selected from
2-amino-6,7,8-trimethoxyquinazolin-4(3H)-one,
6,7,8-Trimethoxy-2-morpholinoquinazolin-4(3H)-one
2-(4-methylpiperazin)-6,7,8-trimethoxyquinazolin-4(3H)-one
tert-Butyl 6,7,8- trimethoxy-4-oxo-3,4 dihydmquinazolin-2-ylcarbomate
2-(diisopropylamino)-6,7,8-trimethoxyquinazolin-4(3H)-one,
2-(dimethylamino)-6,7,8-trimethoxyquinazolin-4(3H)-one,
2-(di-tert-butylamino)-6,7,8-trimethoxyquinazolin-4(3H)-one,
2-(diethylamino)-6,7,8-trimethoxyquinazolin-4(3H)-one,
2-amino-6,7,8-trimethoxy-4-oxo-3,4-dihydroquinazoline-5-carboxamide,
2-(diisopropylamino)-6,7,8-trimethoxy-4-oxo-3,4-dihydroquinazoline-5-carboxamide,
2-(dimethylamino)-6,7,8-trimethoxy-4-oxo-3,4-dihydroquinazoline-5-carboxamide,
2-(di-tert-butylamino)-6,7,8-trimethoxy-4-oxo-3,4-dihydroquinazoline-5-carboxamide,
2-(diethylamino)-6,7,8-trimethoxy-4-oxo-3,4-dihydroquinazoline-5-carboxamide,
2-amino-6,7,8-trihydroxyquinazolin-4(3H)-one,
6,7,8-trihydroxy-2-morpholinoquinazolin-4(3H)-one
6,7,8-trihydroxy-2-(4-methylpiperazin-1-yl)quinazolin-4(3H)-one
tert-Butyl 6,7,8- trihydroxy-4-oxo-3,4-dihydroquinazolin-2-ylcarbomate
2-(diisopropylamino)-6,7,8-trihydroxyquinazolin-4(3H)-one,
2-(dimethylamino)-6,7,8-trihydroxyquinazolin-4(3H)-one,
2-(di-tert-butylamino)-6,7,8-trihydroxyquinazolin-4(3H)-one,
2-(diethylamino)-6,7,8-trihydroxyquinazolin-4(3H)-one,
2-amino-6,7,8-trihydroxy-4-oxo-3,4-dihydroquinazoline-5-carboxamide,
2-(diisopropylamino)-6,7,8-trihydroxy-4-oxo-3,4-dihydroquinazoline-5-carboxamide,
2-(dimethylamino)-6,7,8-trihydroxy-4-oxo-3,4-dihydroquinazoline-5-carboxamide,
2-(di-tert-butylamino)-6,7,8-trihydroxy-4-oxo-3,4-dihydroquinazoline-5-carboxamide or
2-(diethylamino)-6,7,8-trihydroxy-4-oxo-3,4-dihydroquinazoline-5-carboxamide
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

In another preferred embodiment of the invention for the compound according to the invention according to formula II
R¹⁶ represents hydrogen.

In a very preferred embodiment of the invention the compound according to the invention is a compound according to formula III

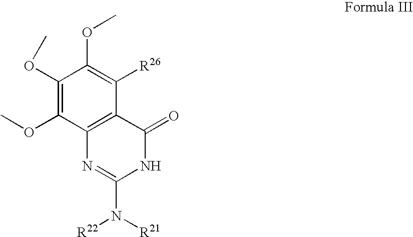

wherein
R²¹ and R²² independently of oneanother represent hydrogen; C₁₋₄-alkyl, with alkyl being linear or branched, saturated or unsaturated, substituted or unsubstituted; or an appropriate protective group;
or R²¹ and R²² together with the Nitrogen they both bind to form a heterocyclic ring or an appropriate protective group;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted; or an appropriate protective group;
or R²⁷ and R²⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.
In a preferred embodiment of the invention for the compound according to the invention according to formula III
R²¹ and R²² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R²¹ and R²² together with the Nitrogen they both bind to form a heterocyclic ring of the following formula:

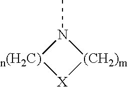

with n being 1, 2, 3 or 4, m being 1, 2, 3 or 4 and (n+m) being ≦6 and X being selected from S, O, NR²⁹ or CHR²⁹ with R²⁹ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R²⁷ and R²⁸ together with the Nitrogen they both bind to form a heterocyclic ring of the following formula:

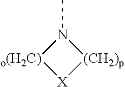

with o being 1, 2, 3 or 4, p being 1, 2, 3 or 4 and (o+p) being ≦6 and Y being selected from S, O, NR³⁰ or CHR³⁰ with R³⁰ being selected from hydrogen or C₁₋₄-alkyl, with alkyl being linear or branched, saturated or unsaturated, unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

In another preferred embodiment of the invention for the compound according to the invention according to formula III
R²¹ and R²² independently of oneanother represent hydrogen; C₁₋₄-alkyl, with alkyl being linear or branched, saturated or unsaturated, substituted or unsubstituted;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted.
In another preferred embodiment of the invention for the compound according to the invention according to formula III
R²¹ and R²² independently of oneanother represent hydrogen; C₁₋₄-alkyl, with alkyl being linear or branched, saturated and unsubstituted; and/or
R²⁶ represents hydrogen or C(O)—NR²⁷R²⁸;
R²⁷ and R²⁸ independently of oneanother represent hydrogen; C₁₋₄-alkyl, with alkyl being linear or branched, saturated and unsubstituted.

In another preferred embodiment of the invention the compound according to the invention according to formula III is selected from
2-amino-6,7,8-trimethoxyquinazolin-4(3H)-one,
6,7,8-Trimethoxy-2-morpholinoquinazolin-4(3H)-one,
2-(4-methylpiperazin)-6,7,8-trimethoxyquinazolin-4(3H)-one,
tert-Butyl 6,7,8- trimethoxy-4-oxo-3,4 dihydroquinazolin-2-ylcarbomate,
2-(diisopropylamino)-6,7,8-trimethoxyquinazolin-4(3H)-one,
2-(dimethylamino)-6,7,8-trimethoxyquinazolin-4(3H)-one,
2-(di-tert-butylamino)-6,7,8-trimethoxyquinazolin-4(3H)-one,
2-(diethylamino)-6,7,8-trimethoxyquinazolin-4(3H)-one,
2-amino-6,7,8-trimethoxy-4-oxo-3,4-dihydroquinazoline-5-carboxamide,
2-(diisopropylamino)-6,7,8-trimethoxy-4-oxo-3,4-dihydroquinazoline-5-carboxamide,
2-(dimethylamino)-6,7,8-trimethoxy-4-oxo-3,4-dihydroquinazoline-5-carboxamide,
2-(di-tert-butylamino)-6,7,8-trimethoxy-4-oxo-3,4-dihydroquinazoline-5-carboxamide or
2-(diethylamino)-6,7,8-trimethoxy-4-oxo-3,4-dihydroquinazoline-5-carboxamide,
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.
   In another preferred embodiment of the invention for the compound according to the invention according to formula III R²⁶ represents hydrogen.

In a very preferred embodiment of the invention the compound according to the invention according to formula III is selected from
2-amino-6,7,8-trimethoxyquinazolin-4(3H)-one,
6,7,8-Trimethoxy-2-morpholinoquinazolin-4(3H)-one,
2-(4-methylpiperazin)-6,7,8-trimethoxyquinazolin-4(3H)-one,
tert-Butyl 6,7,8- trimethoxy-4-oxo-3,4-dihydroquinazolin-2-ylcarbomate,
2-(diisopropylamino)-6,7,8-trimethoxyquinazolin-4(3H)-one,
2-(dimethylamino)-6,7,8-trimethoxyquinazolin-4(3H)-one,
2-(di-tert-butylamino)-6,7,8-trimethoxyquinazolin-4(3H)-one or
2-(diethylamino)-6,7,8-trimethoxyquinazolin-4(3H)-one;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

In a very preferred embodiment of the invention the compound according to the invention is a compound according to formula IIIa

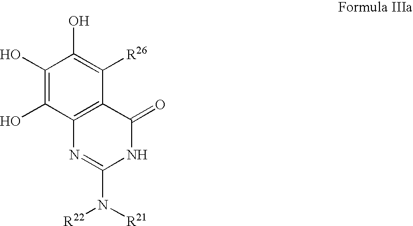

wherein
R²¹ and R²² independently of oneanother represent hydrogen; C₁₋₄-alkyl, with alkyl being linear or branched, saturated or unsaturated, substituted or unsubstituted; or an appropriate protective group;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted; or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.
In a preferred embodiment of the invention for the compound according to the invention according to formula IIIa
R²¹ and R²² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc).
In another preferred embodiment of the invention for the compound according to the invention according to formula IIIa
R²¹ and R²² independently of oneanother represent hydrogen; C₁₋₄-alkyl, with alkyl being linear or branched, saturated or unsaturated, substituted or unsubstituted;
R²⁶ represents hydrogen or C(O)—NR²⁷R²⁸;

R²⁷ and R²⁸ independently of oneanother represent hydrogen; C₁₋₄-alkyl, with alkyl being linear or branched, saturated or unsaturated, substituted or unsubstituted.
In another preferred embodiment of the invention for the compound according to the invention according to formula IIIa
R²¹ and R²² independently of oneanother represent hydrogen; C₁₋₄-alkyl, with alkyl being linear or branched, saturated and unsubstituted; and/or
R²⁶ represents hydrogen or C(O)—NR²⁷R²⁸;

R²⁷ and R²⁸ independently of oneanother represent hydrogen; C₁₋₄-alkyl, with alkyl being linear or branched, saturated and unsubstituted.

In a further preferred embodiment of the invention the compound according to the invention according to formula IIIa is selected from
2-amino-6,7,8-trihydroxyquinazolin-4(3H)-one
6,7,8-trihydroxy-2-morpholinoquinazolin-4(3H)-one
6,7,8-trihydroxy-2-(4-methylpiperazin-1-yl)quinazolin-4(3H)-one
tert-Butyl 6,7,8- trihydroxy-4-oxo-3,4-dihydroquinazolin-2-ylcarbomate
2-(diisopropylamino)-6,7,8-trihydroxyquinazolin-4(3H)-one
2-(dimethylamino)-6,7,8-trihydroxyquinazolin-4(3H)-one
2-(di-tert-butylamino)-6,7,8-trihydroxyquinazolin-4(3H)-one
2-(diethylamino)-6,7,8-trihydroxyquinazolin-4(3H)-one
2-amino-6,7,8-trihydroxy-4-oxo-3,4-dihydroquinazoline-5-carboxamide
2-(diisopropylamino)-6,7,8-trihydroxy-4-oxo-3,4-dihydroquinazoline-5-carboxamide
2-(dimethylamino)-6,7,8-trihydroxy-4-oxo-3,4-dihydroquinazoline-5-carboxamide
2-(di-tert-butylamino)-6,7,8-trihydroxy-4-oxo-3,4-dihydroquinazoline-5-carboxamide
2-(diethylamino)-6,7,8-trihydroxy-4-oxo-3,4-dihydroquinazoline-5-carboxamide
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

In another preferred embodiment of the invention for the compound according to the invention according to formula IIa
R²⁶ represents hydrogen.

In a very preferred embodiment of the invention the compound according to the invention according to formula IIIa is selected from
2-amino-6,7,8-trihydroxyquinazolin-4(3H)-one,
6,7,8-trihydroxy-2-morpholinoquinazolin-4(3H)-one,
6,7,8-trihydroxy-2-(4-methylpiperazin-1-yl)quinazolin-4(3H)-one,
tert-Butyl 6,7,8- trihydroxy-4-oxo-3,4-dihydroquinazolin-2-ylcarbomate
2-(diisopropylamino)-6,7,8-trihydroxyquinazolin-4(3H)-one,
2-(dimethylamino)-6,7,8-trihydroxyquinazolin-4(3H)-one,
2-(di-tert-butylamino)-6,7,8-trihydroxyquinazolin-4(3H)-one or
2-(diethylamino)-6,7,8-trihydroxyquinazolin-4(3H)-one;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

Another preferred aspect of the invention are chemical process especially processes for the production of compounds according to the invention or intermerdiates thereof.

A part of these processes can be seen in the overall process according to Scheme I leading to compounds according to formula I:

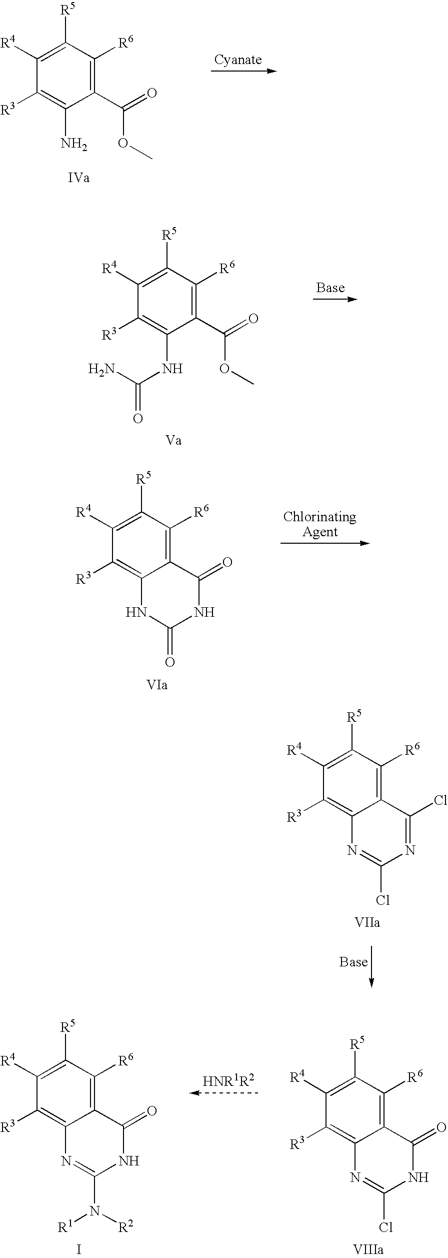

In this overall Scheme I the reactions are carried out in a suitable solvent or reaction medium and R¹, R², R³, R⁴, R⁵ and R⁶ have the meaning mentioned above.

It is preferred that the Cyanate (salt of the cyanic acid) in Scheme I is selected from KOCN or NaOCN, more preferably KOCN.

It is further preferred that the Base in Scheme I is an anorganic base, preferably a hydroxide, especially NaOH or KOH, most preferably NaOH.

It is further preferred that the chlorinating agent in Scheme I is anorganic compound, most preferably POCL₃.

In a majority of the cases R⁶ in IVa in Scheme I is hydrogen with—if applicable—the Amid C(O)NR⁷R⁸ being introduced at some later stage according to reactions well known in the art.

In a preferred embodiment of the invention a compound according to the invention according to formula I is prepared by reacting a compound of formula VIIIa

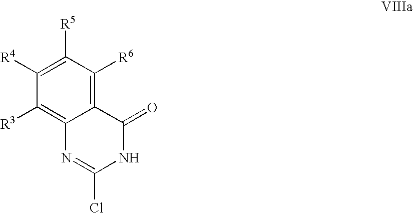

with a secondary amine HNR¹R² in a suitable solvent or reaction medium and R¹, R², R³, R⁴, R⁵ and R⁶ having the meaning mentioned above.

In a preferred embodiment of this process to obtain a compound according to formula I to prepare the abovementioned compound according to formula VIIIa a compound of formula VIIIa

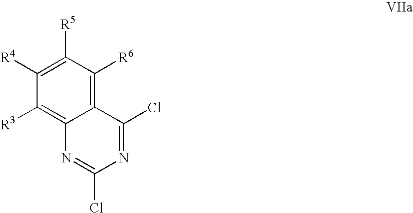

is reacted with a base in a suitable solvent or reaction medium and R¹, R², R³, R⁴, R⁵ and R⁶ having the meaning mentioned above.

Here it is preferred that the Base is an anorganic base, preferably a hydroxide, especially NaOH or KOH, most preferably NaOH.

In a preferred embodiment of this process to obtain a compound according to formula I to prepare the abovementioned compound according to formula VIIIa a compound of formula VIa

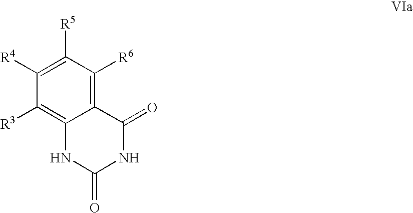

is reacted with a chlorinating agent in a suitable solvent or reaction medium and R³, R⁴, R⁵ and R⁶ having the meaning mentioned above.

Here it is preferred that the chlorinating agent is an anorganic compound, most preferably POCL₃.

In a preferred embodiment of this process to obtain a compound according to formula I to prepare the abovementioned compound according to formula VIa a compound of formula Va

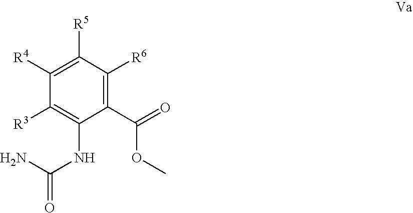

is reacted with a base in a suitable solvent or reaction medium and R³, R⁴, R⁵ and R⁶ having the meaning mentioned above.

Here it is preferred that the Base is an anorganic base, preferably a hydroxide, especially NaOH or KOH, most preferably NaOH.

In a preferred embodiment of this process to obtain a compound according to formula I to prepare the abovementioned compound according to formula Va a compound of formula IVa

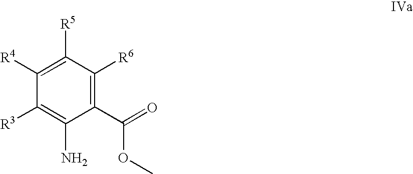

is reacted with a cyanate/salt of cyanic acid in a suitable solvent or reaction medium and R³, R⁴, R⁵ and R⁶ having the meaning mentioned above.

Here it is preferred that the cyanate (salt of the cyanic acid) is selected from KOCN or NaOCN, more preferably KOCN.

A selected part of these processes can be seen in the overall process according to Scheme II leading to compounds according to formula II:

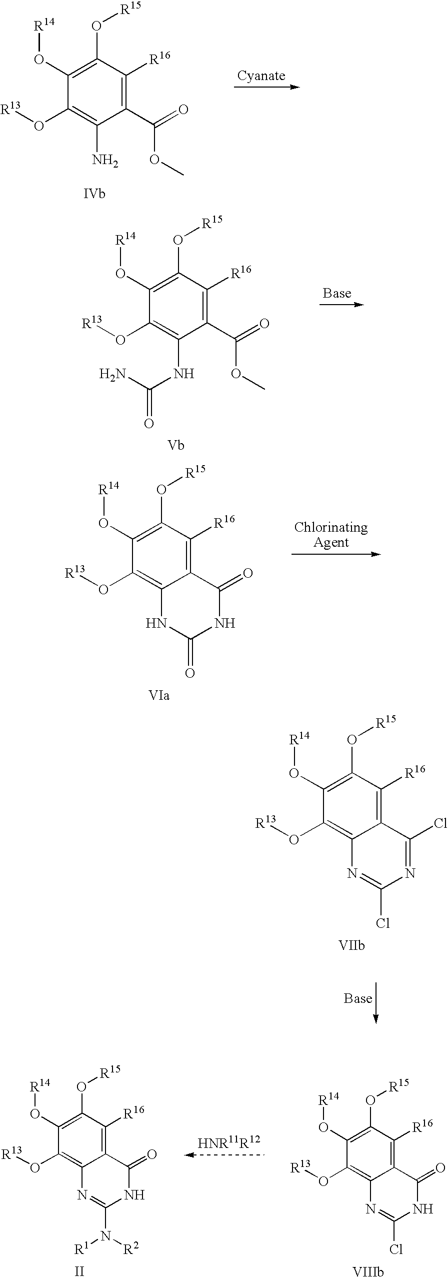

In this overall Scheme II the reactions are carried out in a suitable solvent or reaction medium and R¹¹, R¹², R¹³, R¹⁴, R¹⁵ and R¹⁶ have the meaning mentioned above.

It is preferred that the Cyanate (salt of the cyanic acid) in Scheme II is selected from KOCN or NaOCN, more preferably KOCN.

It is further preferred that the Base in Scheme II is an anorganic base, preferably a hydroxide, especially NaOH or KOH, most preferably NaOH.

It is further preferred that the chlorinating agent in Scheme II is anorganic compound, most preferably POCL₃.

In a majority of the cases R¹⁶ in IVb in Scheme II is hydrogen with—if applicable—the Amid C(O)NR¹⁷R¹⁸ being introduced at some later stage according to reactions well known in the art.

In a preferred embodiment of the invention a compound according to the invention according to formula II is prepared by reacting a compound of formula VIIIb

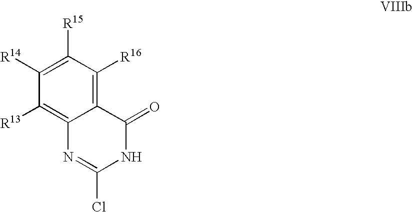

with a secondary amine HNR¹¹R¹² in a suitable solvent or reaction medium and R¹¹, R¹², R¹³, R¹⁴, R¹⁵ and R¹⁶ having the meaning mentioned above.

In a preferred embodiment of this process to obtain a compound according to formula II to prepare the abovementioned compound according to formula VIIIb a compound of formula VIIb

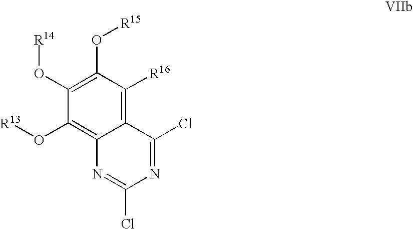

is reacted with a base in a suitable solvent or reaction medium and R¹¹, R¹², R¹³, R¹⁴, R¹⁵ and R¹⁶ having the meaning mentioned above.

Here it is preferred that the base is an anorganic base, preferably a hydroxide, especially NaOH or KOH, most preferably NaOH.

In a preferred embodiment of this process to obtain a compound according to formula II to prepare the abovementioned compound according to formula VIIb a compound of formula VIb

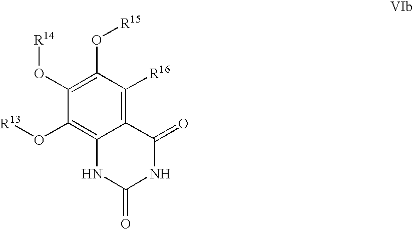

is reacted with a chlorinating agent in a suitable solvent or reaction medium and R¹³, R¹⁴, R¹⁵ and R¹⁶ having the meaning mentioned above.

Here it is preferred that the chlorinating agent is an anorganic compound, most preferably POCL₃.

In a preferred embodiment of this process to obtain a compound according to formula II to prepare the abovementioned compound according to formula VIb a compound of formula Vb

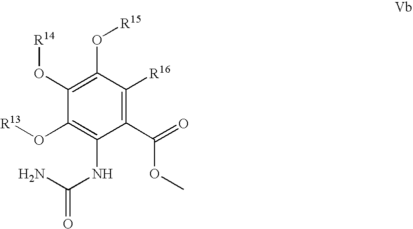

is reacted with a base in a suitable solvent or reaction medium and R¹³, R¹⁴, R¹⁵ and R¹⁶ having the meaning mentioned above.

Here it is preferred that the Base is an anorganic base, preferably a hydroxide, especially NaOH or KOH, most preferably NaOH.

In a preferred embodiment of this process to obtain a compound according to formula II to prepare the abovementioned compound according to formula Vb a compound of formula IVb

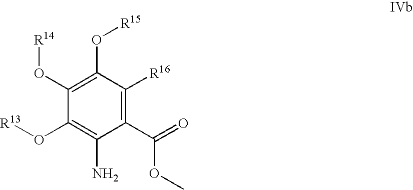

is reacted with a cyanate/salt of cyanic acid in a suitable solvent or reaction medium and R¹³, R¹⁴, R¹⁵ and R¹⁶ having the meaning mentioned above.

Here it is preferred that the cyanate (salt of the cyanic acid) is selected from KOCN or NaOCN, more preferably KOCN.

A further selected part of these processes can be seen in the overall process according to Scheme III leading to compounds according to formula III:

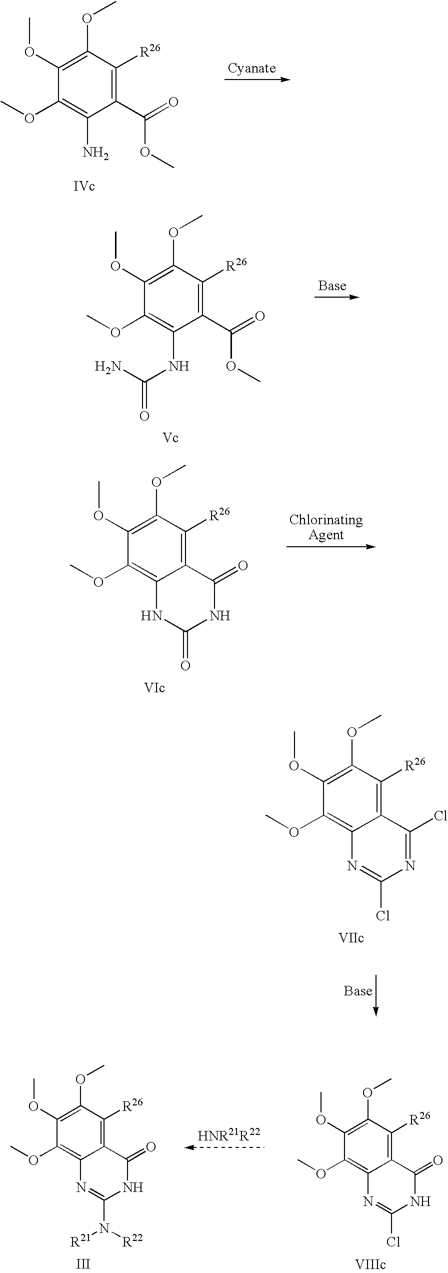

In this overall Scheme III the reactions are carried out in a suitable solvent or reaction medium and R²¹, R²² and R²⁶ have the meaning mentioned above.

It is preferred that the Cyanate (salt of the cyanic acid) in Scheme III is selected from KOCN or NaOCN, more preferably KOCN.

It is further preferred that the Base in Scheme III is an anorganic base, preferably a hydroxide, especially NaOH or KOH, most preferably NaOH.

It is further preferred that the chlorinating agent in Scheme III is anorganic compound, most preferably POCL₃.

In a majority of the cases R²⁶ in IVc in Scheme III is hydrogen with—if applicable—the Amid C(O)NR²⁷R²⁸ being introduced at some later stage according to reactions well known in the art.

In a preferred embodiment of the invention a compound according to the invention according to formula III is prepared by reacting a compound of formula VIIIc

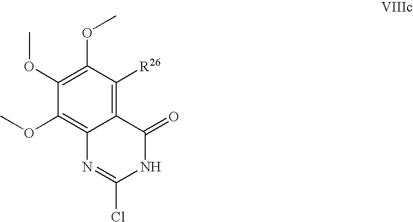

with a secondary amine HNR²¹R²² in a suitable solvent or reaction medium and R²⁶ having the meaning mentioned above.

In a preferred embodiment of this process to obtain a compound according to formula III to prepare the abovementioned compound according to formula VIIIc a compound of formula VIIc

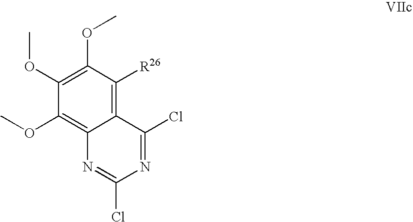

is reacted with a base in a suitable solvent or reaction medium and R²⁶ having the meaning mentioned above.

Here it is preferred that the base is an anorganic base, preferably a hydroxide, especially NaOH or KOH, most preferably NaOH.

In a preferred embodiment of this process to obtain a compound according to formula III to prepare the abovementioned compound according to formula VIIc a compound of formula VIc

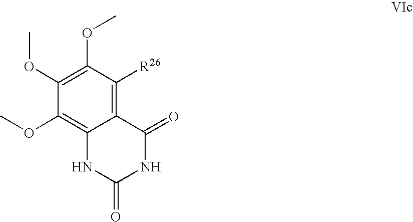

is reacted with a chlorinating agent in a suitable solvent or reaction medium and R²⁶ having the meaning mentioned above.

Here it is preferred that the chlorinating agent is an anorganic compound, most preferably POCL₃.

In a preferred embodiment of this process to obtain a compound according to formula III to prepare the abovementioned compound according to formula VIc a compound of formula Vc

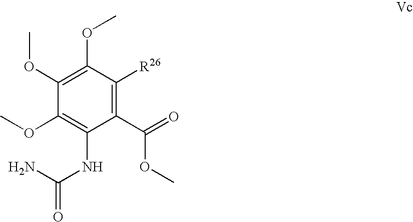

is reacted with a base in a suitable solvent or reaction medium and R²⁶ having the meaning mentioned above.

Here it is preferred that the Base is an anorganic base, preferably a hydroxide, especially NaOH or KOH, most preferably NaOH.

In a preferred embodiment of this process to obtain a compound according to formula III to prepare the abovementioned compound according to formula Vc a compound of formula IVc

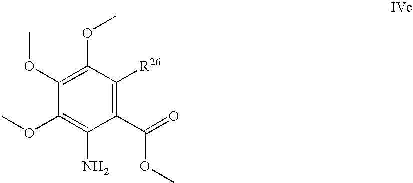

is reacted with a cyanate/salt of cyanic acid in a suitable solvent or reaction medium and R²⁶ having the meaning mentioned above.

Here it is preferred that the cyanate (salt of the cyanic acid) is selected from KOCN or NaOCN, more preferably KOCN.

A further selected part of these processes can be seen in the overall process according to Scheme IIIa leading to compounds according to formula IIIa, based on Scheme III:

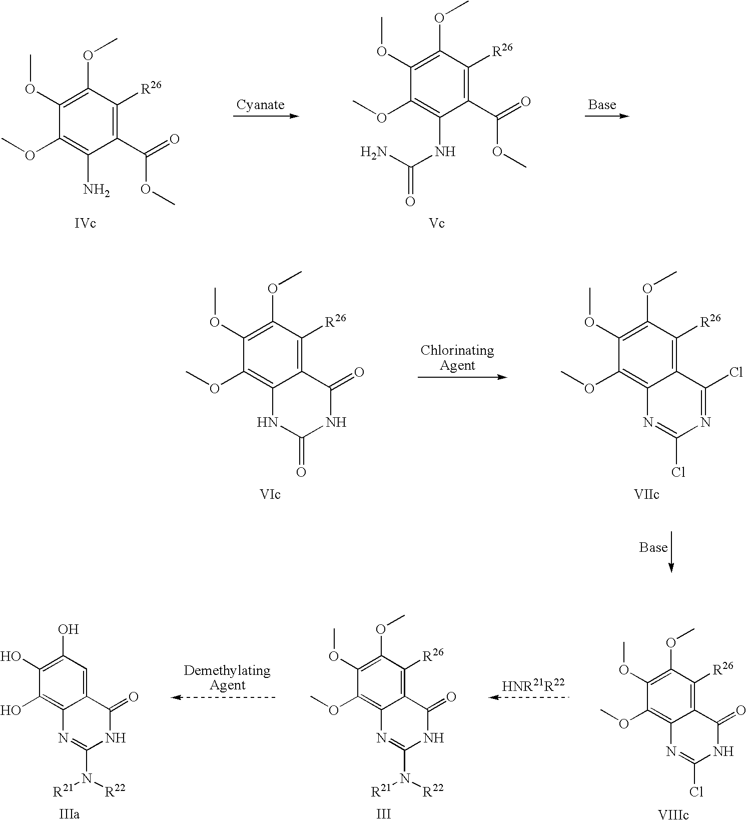

In this overall Scheme IIIa the reactions are carried out in a suitable solvent or reaction medium and R²¹, R²² and R²⁶ have the meaning mentioned above.

It is preferred that the Cyanate (salt of the cyanic acid) in Scheme IIIa is selected from KOCN or NaOCN, more preferably KOCN.

It is further preferred that the Base in Scheme IIIa is an anorganic base, preferably a hydroxide, especially NaOH or KOH, most preferably NaOH.

It is further preferred that the chlorinating agent in Scheme IIIa is anorganic compound, most preferably POCL₃.

It is further preferred that the demethylating agent in Scheme IIIa is BCL₃.

In a majority of the cases R²⁶ in IVc in Scheme IIIa is hydrogen with—if applicable—the Amid C(O)NR²⁷R²⁸ being introduced at some later stage according to reactions well known in the art.

In a preferred embodiment of the invention a compound according to the invention according to formula IIIa is prepared by reacting a compound of formula III

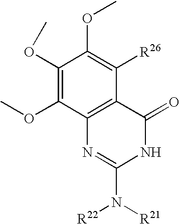

with a demethylating agent in a suitable solvent or reaction medium and R²⁶ having the meaning mentioned above.

Here it is preferred that the demethylating agent is BCL₃.

Another preferred aspect of the invention is the use of at least one compound according to the invention as an intermediate in the synthesis of active biomolecules.

It further seems that the compounds according to the invention surprisingly are sodium channel binders or blockers and thus seem to have pharmaceutical activity (see e.g. Anger et al., J MedChem. Vol. 44, No. 2, (2001) 115-137).

Thus as the compounds according to the invention are toxicologically acceptable they are therefore suitable as pharmaceutical active substances for the preparation of medicaments.

Thus, another aspect of the present invention relates to a Medicament comprising at least one compound according to the invention and optionally one or more pharmaceutically acceptable excipients.

In this application the term medicament should be considered as equal to the term pharmaceutical composition.

The medicament according to the present invention may be in any form suitable for the application to humans and/or animals, preferably humans including infants, children and adults and can be produced by standard procedures known to those skilled in the art. The composition of the medicament may vary depending on the route of administration.

The medicament of the present invention may for example be administered parentally in combination with conventional injectable liquid carriers, such as water or suitable alcohols. Conventional pharmaceutical excipients for injection, such as stabilizing agents, solubilizing agents, and buffers, may be included in such injectable compositions. These medicaments may for example be injected intramuscularly, intraperitoneally, or intravenously.

Solid oral compositions (which are preferred as are liquid ones) may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to the methods well known in normal pharmaceutical practice, in particular with an enteric coating.

The mentioned formulations will be prepared using standard methods such as those described or referred to in the Spanish and US Pharmacopeias and similar reference texts.

Medicaments according to the present invention may also be formulated into orally administrable compositions containing one or more physiologically compatible carriers or excipients, in solid or liquid form. These compositions may contain conventional ingredients such as binding agents, fillers, lubricants, and acceptable wetting agents. The compositions may take any convenient form, such as tablets, pellets, capsules, lozenges, aqueous or oily solutions, suspensions, emulsions, or dry powdered forms suitable for reconstitution with water or other suitable liquid medium before use, for immediate or retarded release.

The liquid oral forms for administration may also contain certain additives such as sweeteners, flavoring, preservatives, and emulsifying agents. Non-aqueous liquid compositions for oral administration may also be formulated, containing edible oils. Such liquid compositions may be conveniently encapsulated in e.g., gelatin capsules in a unit dosage amount.

The compositions of the present invention may also be administered topically or via a suppository.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

Another preferred aspect of the invention is the use of at least one compound according to the invention (and optionally one or more pharmaceutically acceptable excipients), for the preparation/manufacture of a medicament for the treatment of CNS Disorders.

Another preferred aspect of the invention is the use of at least one compound according to the invention (and optionally one or more pharmaceutically acceptable excipients), for the preparation of a medicament for the treatment of pain, especially neuropathic pain, stroke, addiction and epilepsy.

The present invention is illustrated below with the aid of examples and figures. These illustrations are given solely by way of example and do not limit the general spirit of the present invention.

### EXPERIMENTAL PART

### Examples

General Methods and Materials.

All reactions described below were carried out under argon atmosphere unless otherwise noted. The solvents used were distilled and dried under argon atmosphere before use. All starting materials were purchased commercially (Aldrich, Fluka and Merck) and used without further purification. Flash Chromatography was executed on columns loaded with 230-400 mesh silica gel Merck. TLC was carried out on silica gel Merck (Kieselgel 60F-254).

Melting points (mp) were determined on a Reichert Microscopic Hot-Stage. ¹H and ¹³C NMR spectra were measured on a Varian Gemini-200 and a Varian Inova-300 spectrometer with (CH₃)₄Si as an internal reference and CDCl₃ as solvent unless otherwise noted. Both ¹H and ¹³C NMR spectral data are reported in parts per million (δ) relative to residual sign of the solvent (CDCl₃, 7.26 ppm and 77.0 ppm for ¹H and ¹³C NMR, respectively). ¹H and ¹³C NMR designations are: s (singlete); s br. (broad singlete); d (doublete); t (triplete); q (cuartete); m (multiplete). Infrared (IR) spectra were record on a Perkin-Elmer FT-IR spectrometer. UV spectra were record on a Perkin-Elmer 402 spectrometer. Low-resolution mass (LRMS) spectra were obtained on a Hewlett Packard 5973 MSD spectrometer with a direct inlet system (EI) at 70 eV.

The compounds of general formula I were nominated, in general, as derivatives of quinazolin-4(3H)-one and were numerated following the numeration described below.

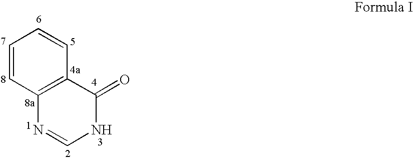

### Example 1

### 2-(diisopropylamino)-6,7,8-trimethoxyquinazolin-4(3H)-one

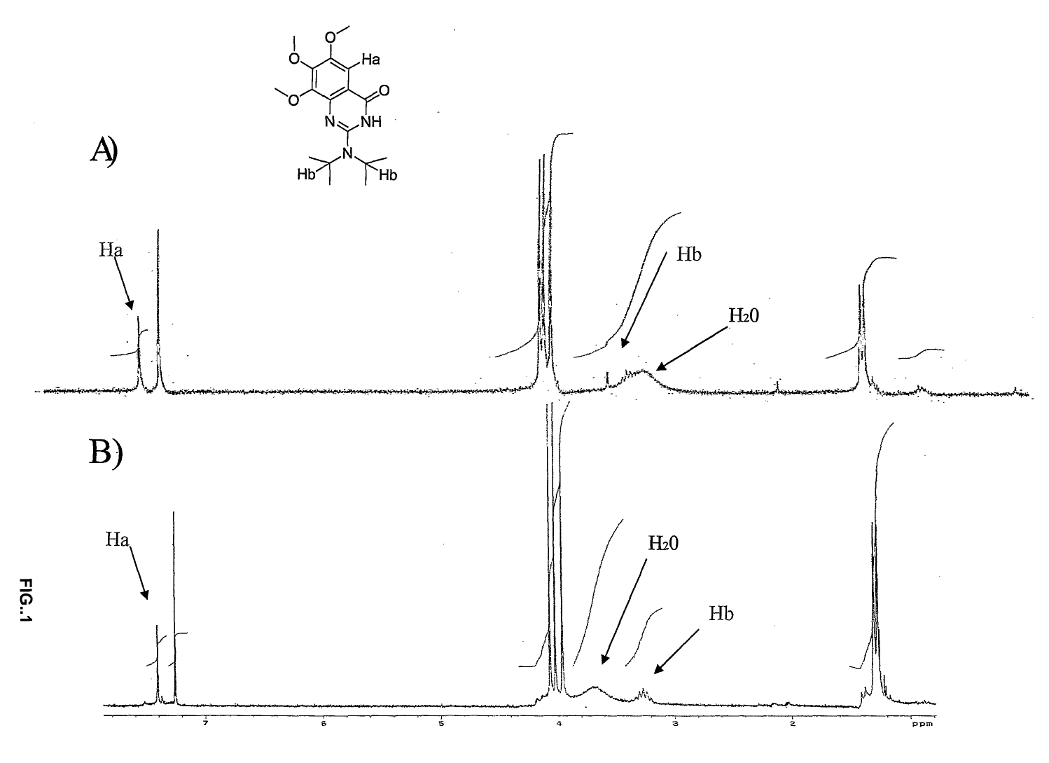

Example 1 was produced according to the following reaction scheme. The NMR spectrum of the resulting compound is shown in FIG. 1.

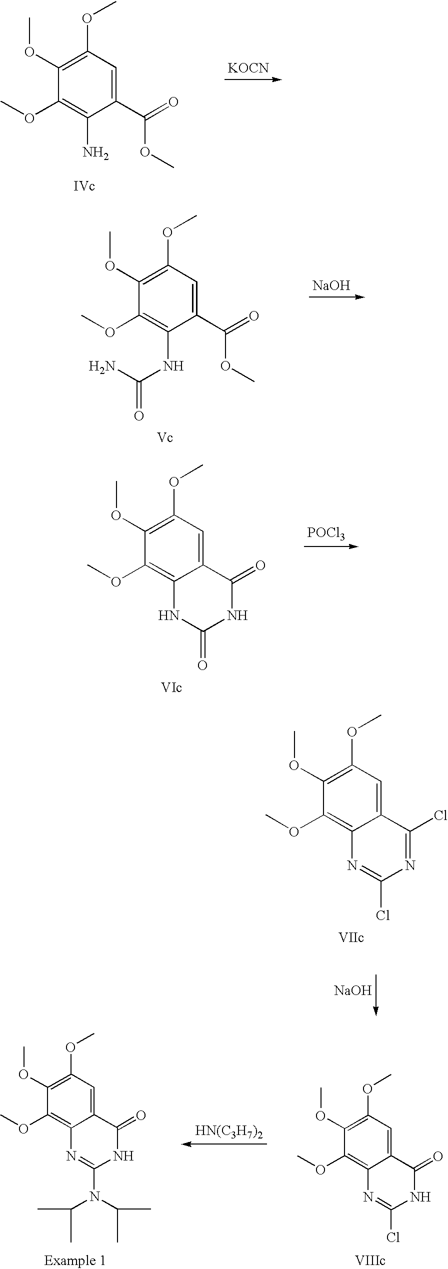

Compound VIc is obtained (white solid, quantitative yield) by the sequence: a) reaction of Compound IVc (Methyl 3,4,5-trimethoxyanthranilate or Benzoic acid, 2-amino-3,4,5-trimethoxy-, methyl ester; commercially available from companies like Merck, Apin or Maybrigde) and potassium cyanate in acetic acid (aqueous solution), and b) treatment of the crude suspension with NaOH (50%).

Compound VIIIc is obtained (white solid, quantitative yield) by the sequence: a) reaction of Compound VIc and phosphours (III) oxychloride in presence of N,N-dimethylaniline, and b) hydrolysis with NaOH (1N) using THF as solvent.

### 2-(diisopropylamino)-6,7,8-trimethoxy-2-morpholinoquinazolin-4(3H)-one

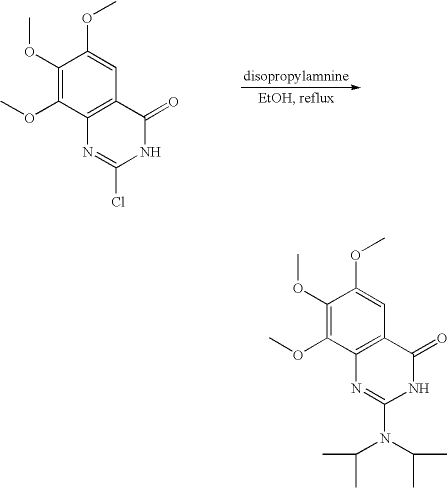

In a Kimble, to a mixture of 2-chloro-6,7,8-trimethoxyquinazolin-4(3H)-one (70 mg, 0.259 mmol) in EtOH (2.5 ml) was added disiopropylamine (0.4 ml, 2.845 mmol). The resulting mixture was stirred at reflux for 24 h and then concentrated under reduced pressure. The residue was triturated with Et₂O to give 2-(diisopropylamino)-6,7,8-trimethoxy-2-morpholinoquinazolin-4(3H)-one (63 mg, 73%) as a white solid.

R_{f}=0.45 (TLC, Hex:AcOEt 1:3); yield, 73%; white solid; ¹H-NMR (200 MHz, CDCl₃): δ 7.41 (1H, s, H-5), 4.07 (3H, s, OCH₃), 4.03 (3H, s, OCH₃), 3.96 (3H, s, OCH₃), 3.30 (2H, m, CH(CH₃)₂), 1.29 (12H, J=6.4 Hz; d, CH(CH₃)₂

### As an alternative analog 2-(dimethylamino)-6,7,8-trimethoxyquinazolin-4(3H)-one

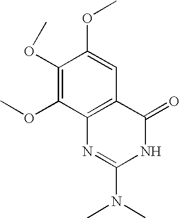

will be produced analogously to example 1 above if HN(CH₃)₂ is added in the last step instead of HN(C₃H₇)₂.

### Example 2

### tert-Butyl 6,7,8- trimethoxy-4-oxo-3,4-dihydroquinazolin-2-ylcarbomate

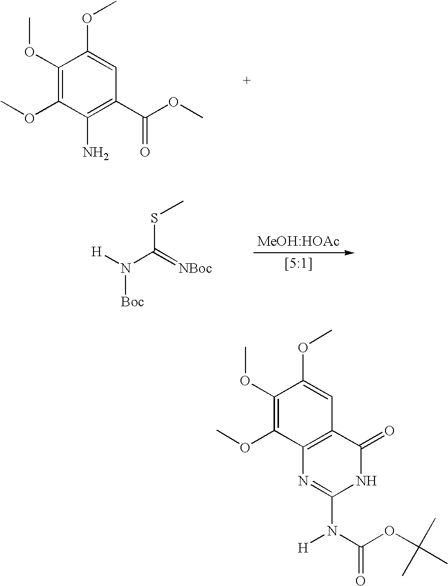

To a solution of methyl 3,4,5-trimethoxy-anthanilate (410 mg, 1.7 mmol) in a mixture 5:1 MeOH/HOAc (30 ml) was added N,N-bis-(tert-butoxycarbony)-2-methyl-2- thiopseudourea (610 mg, 2.1 mmol). The resulting mixture was stirred under argon at room temperature for one day. Then, the mixture was stirred at reflux for another day. To the mixture was added N,N bis-(tert-butoxycarbony)-2-methyl-2- thiopseudourea (508 mg, 1.7 mmol) and was stirred to reflux for another day. Then it was concentrated under reduced pressure. The residue was purified by silica gel column (Hex:AcOEt 3:1) to give tert-Butyl 6,7,8- trimethoxy-4-oxo-3,4-dihydroquinazolin-2-ylcarbomate as a white solid (90 mg, 15%) and methyl 3,4,5-trimethoxy-anthanilate (308 mg, 75%).

R_{f}=0.20 (TLC, Hex: AcOEt); yield, 90%; white solid; ¹H-NMR (200 MHz, CDCl₃): δ 7.40 (1H, s, H-5), 4.01 (3H, s, OCH₃), 3.99 (3H, s, OCH₃), 3.94 (3H, s, OCH₃), 1.54 (9H, s, C(CH₃)₃). (200 MHz, CD₃OD): δ7.37 (1H, s, H-5), 4.84 (1H, s, NH), 3.99 (1H, s, NH), 3.97 (6H, s, OCH₃), 3.93 (3H, s, OCH₃), 1.58 (9H, s, C(CH₃)₃); ¹³C-NMR (50 MHZ, CDCl₃): δ 160.4, 153.4, 151.1, 148.1, 146.4, 145.2, 138.5, 115.1, 102.2, 83.3, 61.7, 61.2, 56.1, 27.8; IR (KBr): v 3436, 2934, 1710, 1681, 1634, 1570, 1466, 1424, 1370, 1250, 1156, 1104 1100, 770 cm⁻¹; LMRS(API-ES⁺): m/z 725 (2M+Na)⁺, 703 (2M+H)⁺, 374 (M+Na)⁺, 352 (M+H)⁺; LMRS(EI): m/z 351 (M⁺, 10), 293 (9), 277 (100), 262 (83), 251 (93), 236 (56), 219 (37), 148 (15).

### Example 3

### 2-amino-6,7,8-trimethoxyquinazolin-4(3H)-one

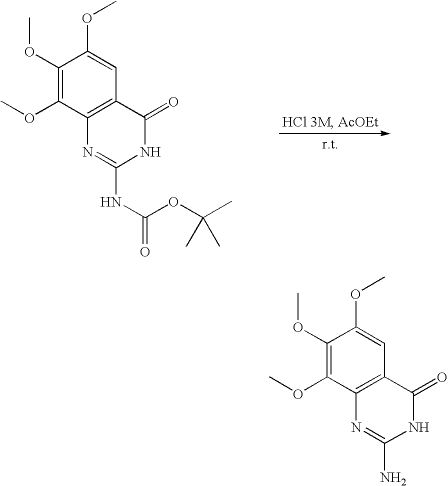

tert-Butyl 6,7,8-trimethoxy-4-oxo-3,4-dihydroquinazolin-2-ylcarbamate (40 mg, 0.114 mmol) as produced according to example 2 was treated with 0.5 ml of a mixture of HCl (3M) and AcOEt (1:1). The mixture was stirred at room temperature for 3.5 h. The solvent was evaporated under reduced pressure. The residue was triturated with Et₂O to give 2-amino-6,7,8-trimethoxyquinazolin-4(3H)-one as a white solid (21 mg, 73%).

Yield, 73%; white solid; ¹H-NMR (300 MHz, CD₃OD): δ 7.38 (1H, s, H-5), 4.05 (3H, s, OCH₃), 4.00 (3H, s, OCH₃), 3.92 (3H, s, OCH₃); ¹³C-NMR (75 MHZ, CD₃OD): δ 160.3, 153.3, 152.5, 149.6, 142.1, 128.3, 111.9, 104.2, 62.4, 61.8, 56.8; IR (KBr): v 3404, 3210, 2949, 1684, 1553, 1500, 1484, 1432, 1277, 1125, 1084, 974 cm⁻¹; LMRS(API-ES⁺): m/z 525 (2M+Na)⁺, 274 (M+Na)⁺, 252 (M+H)⁺.

Alternatively example 3 may also be produced completely analogous to example 1 above, with the exception that in the last step preferably HN(Protect)₂ is used with “Protect” meaning a protective group according to e.g. Greene and Wuts “Protective Groups in Organic Synthsis” 3rd edition, John Wiley & Sons, Inc., p. 573 (1999) (with “Protect” later being removed) instead of HN(C₃H₇)₂ as in example 1.

### Example 4

### 6,7,8-Trimethoxy-2-morpholinoquinazolin-4(3H)-one

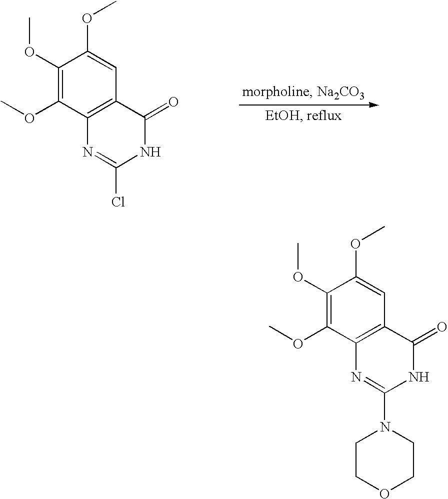

To a mixture of 2-chloro-6,7,8-trimethoxyquinazolin-4(3H)-one (70 mg, 0.259 mmol) (VIIIc produced according to the reaction scheme for example 1) and Na₂CO₃ anhydrous (110 mg, 1.036 mmol) in EtOH (2.6 ml) was added morpholine (0.34 ml, 3.885 mmol). The resulting mixture was stirred at reflux for 3 h and then concentrated under reduced pressure. The residue was purified by silica gel column chromatography (AcOEt) to give 6,7,8-trimethoxy-2-morpholinoquinazolin-4(3H)-one as a white solid (76 mg, 92%).

R_{f}=0.19 (TLC, AcOEt); yield, 92%; white solid; ¹H-NMR (200 MHz, CDCl₃): δ 11.66 (1H, br. s, NH), 7.22 (1H, s, H-5), 4.02 (3H, s, OCH₃), 4.01 (3H, s, OCH₃), 3.90 (3H, s, OCH₃), 3.85 (2H, m, OCH₂CH₂N), 3.76 (2H, m, OCH₂CH₂N); ¹³C-NMR (50 MHz, CDCl₃): δ 164.9, 149.8, 149.2, 148.6, 145.9, 141.0, 112.3, 101.1, 66.4, 61.5, 61.4, 55.9, 45.5; IR (KBr): v 3427, 3130, 3086, 2960, 2840, 1664, 1602, 1472, 1421, 1390, 1307, 1254, 1134, 1114, 1074, 989, 934, 903, 875, 793 cm⁻¹; LRMS (EI): m/z 321 (M⁺, 100), 306 (34), 290 (35), 276 (33), 264 (58), 246 (16), 231 (10), 219 (10), 205 (8), 192 (14); LRMS (API-ES⁺): m/z 665 (2M+Na)⁺, 344 (M+Na)⁺, 322 (M+H)⁺.

### Example 5

### 2-(4-methylpiperazin)-6,7,8-trimethoxyquinazolin-4(3H)-one

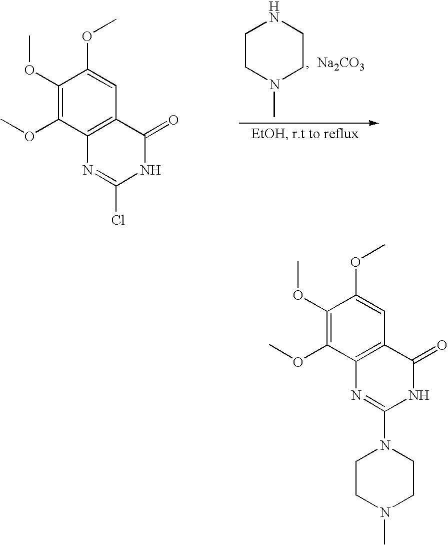

To a stirred solution of 2-chloro-6,7,8-trimethoxyquinazolin-4(3H)-one (120 mg, 0.443 mmol) (VIIIc produced according to the reaction scheme for example 1) and Na₂CO₃ anhydrous (188 mg, 1.772 mmol) in EtOH (4.5 ml) 1-methylpiperazine (0.74 ml, 6.650 mmol) was added. The mixture was heated under reflux during 6.5 hours. After this time, the solvent was evaporated under reduced pressure. The residue was purified by silica gel column chromatography (AcOEt) to give 2-(4-methylpiperazin)-6,7,8-trimethoxyquinazolin-4(3H)-one as a white solid (130 mg, 88%).

R_{f}=0.32 (TLC, MeOH); yield, 88%; white solid; ¹H-NMR (200 MHz, CDCl₃): δ 10.35 (1H, s, NH), 7.25 (1H, s, H-5); 4.02 (3H, s, OCH₃), 4.01 (3H, s, OCH₃), 3.90 (3H, s, OCH₃), 3.73 (4H, t, CH₂, J=4.9 Hz), 2.54 (4H, t, CH₂, J=4.9 Hz), 2.35 (3H, s, NCH₃); ¹³C-NMR (75 MHZ, CDCl₃): δ 164.8, 150.0, 149.3, 149.0, 146.4, 141.8, 112.7, 101.8, 62.0, 61.8, 56.4, 55.0, 46.5, 45.5; IR (KBr): v 3435, 2931, 1669, 1600, 1474, 1417, 1252, 1133, 1079, 1003 cm⁻¹; LMRS(API-ES⁺): m/z 691 (2M+Na)⁺, 357 (M+Na)⁺, 335 (M+H)⁺.

As a follow-up step trihydroxylated compounds may be produced (by treatment with BCl₃) like:
2-(diisopropylamino)-6,7,8-trihydroxyquinazolin-4(3H)-one from example 1
2-(dimethylamino)-6,7,8-trihydroxyquinazolin-4(3H)-one from 2-(dimethylamino)-6,7,8-trimethoxyquinazolin-4(3H)-one (see example 1)
tert-Butyl 6,7,8- trihydroxy-4-oxo-3,4-dihydroquinazolin-2-ylcarbomate from example 2
2-amino-6,7,8-trihydroxyquinazolin-4(3H)-one from example 3, preferably with an appropriate protective group still attached
6,7,8-trihydroxy-2-morpholinoquinazolin-4(3H)-one from example 4,
6,7,8-trihydroxy-2-(4-methylpiperazin-1-yl)quinazolin-4(3H)-one from example 5

1. Substituted quinazoline compound of general formula I, 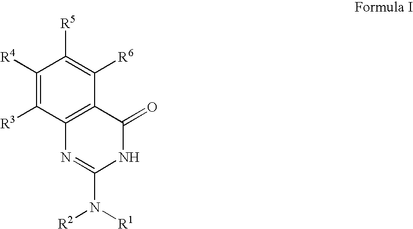
wherein
R¹ and R² independently of oneanother represent hydrogen; C₁₋₄-alkyl, with alkyl being linear or branched, saturated or unsaturated, substituted or unsubstituted; or an appropriate protective group;
or R¹ and R² together with the Nitrogen they both bind to form a heterocyclic ring or an appropriate protective group;
R³ represents halogen, OH or O—C₁₋₄-alkyl, with alkyl being linear or branched, saturated or unsaturated, substituted or unsubstituted; or O—P, with P being an appropriate protective group;
at least one of R⁴ and R⁵ represents halogen; OH; O—C₁₋₄-alkyl, with alkyl
being linear or branched, saturated or unsaturated, substituted or unsubstituted; or O—P, with P being an appropriate protective group;
while the other represents hydrogen; OH; halogen; O—C₁₋₄-alkyl, with alkyl being linear or branched, saturated or unsaturated, substituted or unsubstituted; or O—P, with P being an appropriate protective group;
R⁶ represents hydrogen or C(O)—NR⁷R⁸;
R⁷ and R⁸ independently of oneanother represent hydrogen;
C₁₋₄-alkyl, with alkyl being linear or branched, saturated or unsaturated, substituted or unsubstituted; or an appropriate protective group;
or R⁷ and R⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

2. Compound according to claim 1, characterized in that
R¹ and R² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc),
benzyloxycarbonyl (Cbz), trifluoroacetyl (TFA) or 9-fluorenylmethoxycarbonyl (Fmoc);
or R¹ and R² together with the Nitrogen they both bind to form a heterocyclic ring of the following formula: 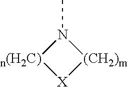
with n being 1, 2, 3 or 4, m being 1, 2, 3 or 4 and (n+m) being ≦6 and X being selected from S, O, NR⁹ or CHR⁹ with R⁹ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R³ represents halogen, OH or O—C₁₋₄-alkyl, with alkyl being linear or branched, saturated or unsaturated,
unsubstituted or substituted by F, Cl, Br, I, NH₂, SH or OH; or O—P, with P being an appropriate protective group selected from tert-butyl, acetyl or benzoyl;
at least one of R⁴ and R⁵ represents halogen; OH; O—C₁₋₄-alkyl, with alkyl
being linear or branched, saturated or unsaturated, unsubstituted or substituted by F, Cl, Br, I, NH₂, SH or OH; or O—P, with P being an appropriate protective group, selected from tert-butyl, acetyl or benzoyl; while the other represents hydrogen; OH; halogen; O—C₁₋₄-alkyl, with alkyl being linear or branched, saturated or unsaturated,
unsubstituted or substituted by F, Cl, Br, I, NH₂, SH or OH; or O—P, with P being an appropriate protective group, selected from tert-butyl, acetyl or benzoyl.
R⁶ represents hydrogen or C(O)—NR⁷R⁸;
R⁷ and R⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R⁷ and R⁸ together with the Nitrogen they both bind to form a heterocyclic ring of the following formula: 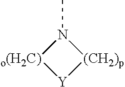
with o being 1, 2, 3 or 4, p being 1, 2, 3 or 4 and (o+p) being ≦6 and Y being selected from S, O, NR¹⁰ or CHR¹⁰ with R¹⁰ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

3. Compound according to claim 1, characterized in that
R¹ and R² independently of oneanother represent hydrogen; C₁₋₄-alkyl, with alkyl being linear or branched, saturated or unsaturated, substituted or unsubstituted;
R³ represents halogen, OH or O—C₁₋₄-alkyl, with alkyl being linear or branched, saturated or unsaturated, substituted or unsubstituted;
at least one of R⁴ and R⁵ represents halogen; OH; O—C₁₋₄-alkyl, with alkyl
being linear or branched, saturated or unsaturated, substituted or unsubstituted;
while the other represents hydrogen; OH; halogen; O—C₁₋₄-alkyl, with alkyl being linear or branched, saturated or unsaturated, substituted or unsubstituted;
R⁶ represents hydrogen or C(O)—NR⁷R⁸;
R⁷ and R⁸ independently of oneanother represent hydrogen; C₁₋₄-alkyl, with alkyl being linear or branched, saturated or unsaturated, substituted or unsubstituted.

4. Compound according to claim 1, characterized in that
R¹ and R² independently of oneanother represent hydrogen; C₁₋₄-alkyl, with alkyl being linear or branched, saturated and unsubstituted; and/or
R³ represents halogen; OH or O—C₁₋₄-alkyl, with alkyl being linear or branched, saturated and unsubstituted; and/or
at least one of R⁴ and R⁵ represents halogen; OH; O—C₁₋₄-alkyl, with alkyl
being linear or branched, saturated and unsubstituted;
while the other represents hydrogen; OH; halogen; O—C₁₋₄-alkyl, with alkyl being linear or branched, saturated and unsubstituted; and/or
R⁶ represents hydrogen or C(O)—NR⁷R⁸;
R⁷ and R⁸ independently of oneanother represent hydrogen; C₁₋₄-alkyl, with alkyl being linear or branched, saturated and unsubstituted.

5. Compound according to claim 1, characterized in that halogen is Cl or F.

6. Compound according to claim 1 according to formula II 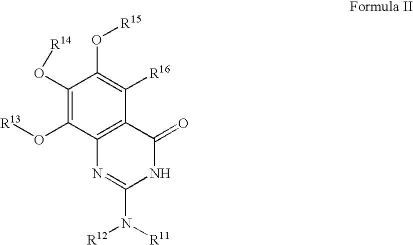
wherein
R¹¹ and R¹² independently of oneanother represent hydrogen; C₁₋₄-alkyl, with alkyl being linear or branched, saturated or unsaturated, substituted or unsubstituted; or an appropriate protective group;
or R¹¹ and R¹² together with the Nitrogen they both bind to form a heterocyclic ring or an appropriate protective group;
R¹³, R¹⁴ and R¹⁵ independently of oneanother represent hydrogen; C₁₋₄-alkyl, with alkyl being linear or branched, saturated or unsaturated, substituted or unsubstituted; or an appropriate protective group;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, substituted or unsubstituted; or an appropriate protective group;
or R¹⁷ and R¹⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

7. Compound according to claim 6, characterized in that
R¹¹ and R¹² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R¹¹ and R¹² together with the Nitrogen they both bind to form a heterocyclic ring of the following formula: 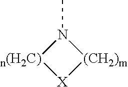
with n being 1, 2, 3 or 4, m being 1, 2, 3 or 4 and (n+m) being ≦6 and X being selected from S, O, NR¹⁹ or CHR¹⁹ with R¹⁹ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R¹³, R¹⁴ and R¹⁵ independently of oneanother represent hydrogen or C₁₋₄-alkyl, with alkyl being linear or branched, saturated or unsaturated, unsubstituted or substituted by F, Cl, Br, I, NH₂, SH or OH; or an appropriate protective group selected from tert-butyl, acetyl or benzoyl;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R¹⁷ and R¹⁸ together with the Nitrogen they both bind to form a heterocyclic ring of the following formula: 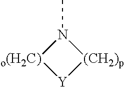
with o being 1, 2, 3 or 4, p being 1, 2, 3 or 4 and (o+p) being ≦6 and Y being selected from S, O, NR²⁰ or CHR²⁰ with R²⁰ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

8. Compound according to claim 6, characterized in that
R¹¹ and R¹² independently of oneanother represent hydrogen; C₁₋₄-alkyl, with alkyl being linear or branched, saturated or unsaturated, substituted or unsubstituted;
R¹³, R¹⁴ and R¹⁵ independently of oneantother represent hydrogen; C₁₋₄-alkyl, with alkyl being linear or branched, saturated or unsaturated, substituted or unsubstituted;
R¹⁶ represents hydrogen or C(O)—NR¹⁷R¹⁸;
R¹⁷ and R¹⁸ independently of oneanother represent hydrogen; C₁₋₄-alkyl, with alkyl being linear or branched, saturated or unsaturated, substituted or unsubstituted.

9. Compound according to claim 6, characterized in that
R¹¹ and R¹² independently of oneanother represent hydrogen; C₁₋₄-alkyl, with alkyl being linear or branched, saturated and unsubstituted; and/or
R¹³, R¹⁴ and R¹⁵ independently of oneantother represent hydrogen; C₁₋₄-alkyl, with alkyl being linear or branched, saturated and unsubstituted; and/or
R¹⁶ represents hydrogen or C(O)—NR¹⁷R¹⁸;
with R¹⁷ and R¹⁸ independently of oneanother represent hydrogen; C₁₋₄-alkyl, with alkyl being linear or branched, saturated and unsubstituted.

10. Compound according to claim 6, characterized in that
R¹³, R¹⁴ and R¹⁵ independently of oneanother represent hydrogen or methyl,
preferably that
R¹³, R¹⁴ and R¹⁵ all represent hydrogen, or
R¹³, R¹⁴ and R¹⁵ all represent methyl.

11. Compound according to claim 6, selected from
2-amino-6,7,8-trimethoxyquinazolin-4(3H)-one,
6,7,8-Trimethoxy-2-morpholinoquinazolin-4(3H)-one
2-(4-methylpiperazin)-6,7,8-trimethoxyquinazolin-4(3H)-one
tert-Butyl 6,7,8- trimethoxy-4-oxo-3,4-dihydroquinazolin-2-ylcarbomate
2-(diisopropylamino)-6,7,8-trimethoxyquinazolin-4(3H)-one,
2-(dimethylamino)-6,7,8-trimethoxyquinazolin-4(3H)-one,
2-(di-tert-butylamino)-6,7,8-trimethoxyquinazolin-4(3H)-one,
2-(diethylamino)-6,7,8-trimethoxyquinazolin-4(3H)-one,
2-amino-6,7,8-trimethoxy-4-oxo-3,4-dihydroquinazoline-5-carboxamide,
2-(diisopropylamino)-6,7,8-trimethoxy-4-oxo-3,4-dihydroquinazoline-5-carboxamide,
2-(dimethylamino)-6,7,8-trimethoxy-4-oxo-3,4-dihydroquinazoline-5-carboxamide,
2-(di-tert-butylamino)-6,7,8-trimethoxy-4-oxo-3,4-dihydroquinazoline-5-carboxamide,
2-(diethylamino)-6,7,8-trimethoxy-4-oxo-3,4-dihydroquinazoline-5-carboxamide,
2-amino-6,7,8-trihydroxyquinazolin-4(3H)-one,
6,7,8-trihydroxy-2-morpholinoquinazolin-4(3H)-one
6,7,8-trihydroxy-2-(4-methylpiperazin-1-yl)quinazolin-4(3H)-one
tert-Butyl 6,7,8- trihydroxy-4-oxo-3,4-dihydroquinazolin-2-ylcarbomate
2-(diisopropylamino)-6,7,8-trihydroxyquinazolin-4(3H)-one,
2-(dimethylamino)-6,7,8-trihydroxyquinazolin-4(3H)-one,
2-(di-tert-butylamino)-6,7,8-trihydroxyquinazolin-4(3H)-one,
2-(diethylamino)-6,7,8-trihydroxyquinazolin-4(3H)-one,
2-amino-6,7,8-trihydroxy-4-oxo-3,4-dihydroquinazoline-5-carboxamide,
2-(diisopropylamino)-6,7,8-trihydroxy-4-oxo-3,4-dihydroquinazoline-5-carboxamide,
2-(dimethylamino)-6,7,8-trihydroxy-4-oxo-3,4-dihydroquinazoline-5-carboxamide,
2-(di-tert-butylamino)-6,7,8-trihydroxy-4-oxo-3,4-dihydroquinazoline-5-carboxamide or
2-(diethylamino)-6,7,8-trihydroxy-4-oxo-3,4-dihydroquinazoline-5-carboxamide
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

12. Compound according to claim 6, characterized in that
R¹⁶ represents hydrogen.

13. Compound according to claim 1 according to formula III 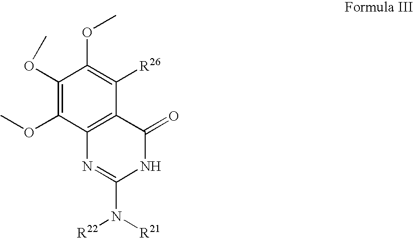
wherein
R²¹ and R²² independently of oneanother represent hydrogen; C₁₋₄-alkyl, with alkyl being linear or branched, saturated or unsaturated, substituted or unsubstituted; or an appropriate protective group;
or R²¹ and R²² together with the Nitrogen they both bind to form a heterocyclic ring or an appropriate protective group;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted; or an appropriate protective group;
or R²⁷ and R²⁸ together with the Nitrogen they both bind to form a heterocyclic ring or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

14. Compound according to claim 13, characterized in that
R²¹ and R²² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R²¹ and R²² together with the Nitrogen they both bind to form a heterocyclic ring of the following formula: 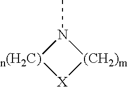
with n being 1, 2, 3 or 4, m being 1, 2, 3 or 4 and (n+m) being ≦6 and X being selected from S, O, NR²⁹ or CHR²⁹ with R²⁹ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP);
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
or R²⁷ and R²⁸ together with the Nitrogen they both bind to
form a heterocyclic ring of the following formula: 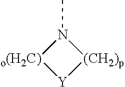
with o being 1, 2, 3 or 4, p being 1, 2, 3 or 4 and (o+p) being ≦6 and Y being selected from S, O, NR³⁰ or CHR³⁰ with R³⁰ being selected from hydrogen or C₁₋₄-alkyl, with alkyl being linear or branched, saturated or unsaturated,
unsubstituted or substituted by F, Cl, Br, I, NH₂, SH or OH;
or an appropriate protective group, selected from phthaloyl (phtalimide), N-1,1,4,4-Tetramethyldisilylazacyclopentane adduct (STABASE), 1,1,3,3-Tetramethyl-1,3-disilaisoindoline (Benzo-STABASE, BSB), N-2,5-bis(triisopropylsilox)pyrrol (BIPSOP).

15. Compound according to claim 13, characterized in that
R²¹ and R²² independently of oneanother represent hydrogen; C₁₋₄-alkyl, with alkyl being linear or branched, saturated or unsaturated, substituted or unsubstituted;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted.

16. Compound according to claim 13, characterized in that
R²¹ and R²² independently of oneanother represent hydrogen; C₁₋₄-alkyl, with alkyl being linear or branched, saturated and unsubstituted; and/or
R²⁶ represents hydrogen or C(O)—NR²⁷R²⁸;
R²⁷ and R²⁸ independently of oneanother represent hydrogen; C₁₋₄-alkyl, with alkyl being linear or branched, saturated and unsubstituted.

17. (canceled)

18. Compound according to claim 13, characterized in that
R² represents hydrogen.

19. Compound according to claim 18 selected from
2-amino-6,7,8-trimethoxyquinazolin-4(3H)-one,
6,7,8-Trimethoxy-2-morpholinoquinazolin-4(3H)-one,
2-(4-methylpiperazin)-6,7,8-trimethoxyquinazolin-4(3H)-one,
tert-Butyl 6,7,8- trimethoxy-4-oxo-3,4-dihydroquinazolin-2-ylcarbomate,
2-(diisopropylamino)-6,7,8-trimethoxyquinazolin-4(3H)-one,
2-(dimethylamino)-6,7,8-trimethoxyquinazolin-4(3H)-one,
2-(di-tert-butylamino)-6,7,8-trimethoxyquinazolin-4(3H)-one or
2-(diethylamino)-6,7,8-trimethoxyquinazolin-4(3H)-one;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

20. Compound according to claim 1 according to formula IIIa 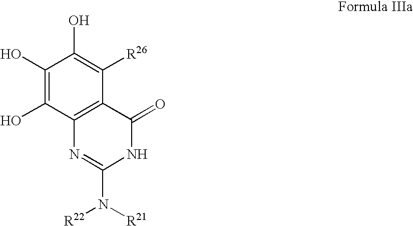
wherein
R²¹ and R²² independently of oneanother represent hydrogen;
C₁₋₄-alkyl, with alkyl being linear or branched, saturated or unsaturated, substituted or unsubstituted; or an appropriate protective group;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted; or an appropriate protective group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

21. Compound according to claim 20, characterized in that
R² and R²² independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc);
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, unsubstituted or substituted by F, Cl, Br, I, NH₂, SH or OH; or an appropriate protective group selected from tert-butoxycarbonyl (Boc), benzyloxycarbonyl (Cbz), trifluoroacetyl (TFA) or 9-fluorenylmethoxycarbonyl (Fmoc).

22. Compound according to claim 20, characterized in that
R²¹ and R²² independently of oneanother represent hydrogen; C₁₋₄-alkyl, with alkyl being linear or branched, saturated or unsaturated, substituted or unsubstituted;
R²⁶ represents hydrogen or C(O)—NR²⁷R²⁸;
R²⁷ and R²⁸ independently of oneanother represent hydrogen; C₁₋₄-alkyl, with alkyl being linear or branched, saturated or unsaturated, substituted or unsubstituted.

23. Compound according to claim 20, characterized in that
R²¹ and R²² independently of oneanother represent hydrogen; C₁₋₄-alkyl, with alkyl being linear or branched, saturated and unsubstituted; and/or
R²⁶ represents hydrogen or C(O)—NR²⁷R²⁸;
R²⁷ and R²⁸ independently of oneanother represent hydrogen; C₁₋₄-alkyl, with alkyl being linear or branched, saturated and unsubstituted.

24. Compound according to claim 20, selected from
2-amino-6,7,8-trihydroxyquinazolin-4(3H)-one
6,7,8-trihydroxy-2-morpholinoquinazolin-4(3H)-one
6,7,8-trihydroxy-2-(4-methylpiperazin-1-yl)quinazolin-4(3H)-one
tert-Butyl 6,7,8- trihydroxy-4-oxo-3,4-dihydroquinazolin-2-ylcarbomate
2-(diisopropylamino)-6,7,8-trihydroxyquinazolin-4(3H)-one
2-(dimethylamino)-6,7,8-trihydroxyquinazolin-4(3H)-one
2-(di-tert-butylamino)-6,7,8-trihydroxyquinazolin-4(3H)-one
2-(diethylamino)-6,7,8-trihydroxyquinazolin-4(3H)-one
2-amino-6,7,8-trihydroxy-4-oxo-3,4-dihydroquinazoline-5-carboxamide
2-(diisopropylamino)-6,7,8-trihydroxy-4-oxo-3,4-dihydroquinazoline-5-carboxamide
2-(dimethylamino)-6,7,8-trihydroxy-4-oxo-3,4-dihydroquinazoline-5-carboxamide
2-(di-tert-butylamino)-6,7,8-trihydroxy-4-oxo-3,4-dihydroquinazoline-5-carboxamide
2-(diethylamino)-6,7,8-trihydroxy-4-oxo-3,4-dihydroquinazoline-5-carboxamide
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

25. Compound according to claim 20, characterized in that
R² represents hydrogen.

26. Compound according to claim 25, selected from
2-amino-6,7,8-trihydroxyquinazolin-4(3H)-one,
6,7,8-trihydroxy-2-morpholinoquinazolin-4(3H)-one,
6,7,8-trihydroxy-2-(4-methylpiperazin-1-yl)quinazolin-4(3H)-one,
tert-Butyl 6,7,8- trihydroxy-4-oxo-3,4-dihydroquinazolin-2-ylcarbomate
2-(diisopropylamino)-6,7,8-trihydroxyquinazolin-4(3H)-one,
2-(dimethylamino)-6,7,8-trihydroxyquinazolin-4(3H)-one,
2-(di-tert-butylamino)-6,7,8-trihydroxyquinazolin-4(3H)-one or
2-(diethylamino)-6,7,8-trihydroxyquinazolin-4(3H)-one;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, or a corresponding salt thereof, or a corresponding solvate thereof.

27. Process for the production of a compound according to claim 1 comprising reacting a compound of formula VIIIa 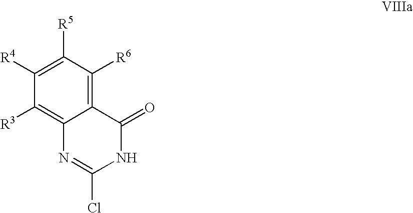
with a secondary amine HNR¹R² in a suitable solvent or reaction medium and R¹, R², R³, R⁴, R⁵ and R⁶ having the meaning according to any of claims 1 to 4.

28-56. (canceled)

57. Medicament comprising at least one compound according to claim 1 and one or more pharmaceutically acceptable excipients.

58. Use of a compound according to claim 1 for the preparation of a medicament for the treatment of CNS disorders.

59. Use of a compound according to claim 1 for the preparation of a medicament for the treatment of pain, stroke, addiction or epilepsy.
